(19) United States
(12) Patent Application Publication
(10) Pub. No.: US 2010/0099131 A1
(43) Pub. Date: Apr. 22, 2010

(54) SOLUTION OF MATRIX

(76) Inventor: Wataru Hattori, Tokyo (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(21) Appl. No.: 12/449,723

(22) PCT Filed: Feb. 13, 2008
(86) PCT No.: PCT/JP2008/000212

§ 371 Date: Aug. 24, 2009

(30) Foreign Application Priority Data
Feb. 28, 2007 (JP) 2007-050429

(51) Int. Cl. C12Q 1/02

(52) U.S. Cl. 435/29

(57) ABSTRACT

A solution of a matrix contains a liquid mixture consists of acetonitrile and water as main solvent component, wherein a volumetric ratio of acetonitrile and water is within a range of from acetonitrile 6.5: water 3.5 to acetonitrile 8: water 2, and wherein sinapinic acid is contained as the matrix.

### TECHNICAL FIELD

The present invention relates to a solution of a matrix for being discharged through a small size nozzle.

### BACKGROUND ART

In recent years, developments in mass spectrometric imaging technology with matrix-assisted laser desorption ionization mass spectrometry (MALDI-MS) are actively conducted. The mass spectrometric imaging is a technology for allowing laser beam to impinge on tissue slices or specimens isolated with chips or gel at the respective positions to conduct a mass spectrometry for each of the impinging locations. Such technology requires the procedures, in which a reagent for accelerating an ionization of the specimen such as protein and the like, which is referred to as “matrix”, is added to the specimen before the impingement with laser beam to present a condition of a crystallization of the specimens with the matrix. Similarly as in the general imaging technologies, higher positional resolution is more preferable in such technology, though the positional resolution is limited by the following factors.

The first factor is a diameter of a spot of an impinging laser beam. The diameter of the laser beam spot in general MALDI-MS is around 100 microns to 200 microns.

The second factor is an unevenness of a specimen distribution in case of adding the matrix. The matrix is ordinarily dissolved in a mixed solvent of a volatile organic solvent (acetonitrile) and water to be added in tissue slices or a chip substrate in a form of a solution, and a specimen such as a protein presented there is taken when the matrix is precipitated or crystallized due to a dehydration of a solvent. On such occasion, an excessive amount of solution for the matrix causes that the matrix solution containing the specimen such as protein and the like is extensively spread over the tissue slice, over the chip or the gel, causing a disturbance on the positional information of specimen such as protein. Therefore, the technology for adding the matrix requires a technology for adding a smaller amount of a solution for every smaller area.

Consequently, in order to reduce a size of a droplet in a mass spectrometric imaging, a small size nozzle such as, for example, an ink-jet, a dispenser, a spray, an electrospray, an ultrasonic atomizer device and the like is employed to add the matrix. In such case, it is desired to have a diameter of a droplet or an inner diameter of a small size nozzle to be substantially equivalent to, or smaller than, a spot diameter of laser beam for the MALDI-MS. In such configuration, a quantity of a droplet of the matrix solution is in the level of nano-liter (nL).

On the other hand, it is required to have sufficiently higher concentration of the the matrix in the solution. It is assumed to be desirable that a crystallization is achieved with the matrix at a mixing ratio of about 1,000 folds of the mixing ratio of a specimen such as protein, peptide and the like in molar ratio.

Sinapinic acid is versatilely employed for the matrix, not only in the case of employing for the imaging technology, but also in the case of conducting an ordinary mass spectrometry. Sinapinic acid is often utilized in a solution in the condition of super-saturation or at a concentration of 10 mg/mL, which is closer to the concentration of the saturated solution, according to a recommendation of manufacturers for mass spectrometers or reagents. A solvent of a liquid mixture of acetonitrile 3: water 7 in a volumetric ratio is often employed, and such solvent often contains trifluoro acetic acid (TFA) at a concentration of 0.1 to 1%.

Molecular weight of sinapinic acid is 224.21, and thus an amount of a substance of sinapinic acid contained in 1 nL of a matrix solution of sinapinic acid at a concentration of 10 mg/mL is 44.6 pmol. Therefore, an amount of a specimen such as protein or peptide corresponding to the molar ratio of one-1,000th is 44.6 fmol. Given that the lower limit of the sensibility for the ordinary commercially available mass spectrometer is 10 fmol, such specimen concentration is almost the value of the lower limit for stable detection.

In other words, it is essential to have a matrix solution concentration of at least about 10 mg/mL for applying thereof to the imaging technology. Actually, a number of researcher's groups who study the mass spectrometric imaging employ such matrix solution of the ordinary condition. For example, Patent Document 1 discloses results of mass spectrometric imaging by employing a matrix solution containing sinapinic acid dissolved at a concentration of 20 mg/mL in a solvent containing 1 acetonitrile and 1 water in volumetric ratio and also containing TFA at a concentration of 0.1 to 1%.

### [Patent Document 1]

Japanese Patent Laid-Open No. 2006-337,371

### DISCLOSURE OF THE INVENTION

### Problem to be Solved by the Invention

However, when the conventional matrix solution is employed for the mass spectrometric imaging, the following problem is caused. The conventional matrix solution is a saturated solution of sinapinic acid or a solution at a concentration extremely closer to the saturation. Therefore, when the matrix solution is discharged from a small size nozzle, sinapinic acid precipitates and crystallizes in the inside of the small size nozzle or at the discharging opening of the nozzle to cause a problem of plugging the small size nozzle.

### Means for Solving Problem

According to an exemplary aspect of the invention, there is provided a solution of a matrix for being discharged through a small size nozzle, containing a liquid mixture consisting of acetonitrile and water as main solvent component, wherein a volumetric ratio of acetonitrile and water is within a range of from acetonitrile 6.5: water 3.5 to acetonitrile 8: water 2, and wherein sinapinic acid is contained as the matrix.

According to another exemplary aspect of the in the matrix solution containing sinapinic acid dissolved therein is selected to be within a range of from acetonitrile 6.5: water 3.5 to acetonitrile 8: water 2, so that the matrix solution containing sinapinic acid at higher concentration can be obtained. In addition, according to the present invention, the matrix solution can be discharged through a small size nozzle without precipitating sinapinic acid therein. Therefore, an addition of the matrix solution in a form of smaller droplet can be conducted, thereby achieving a mass spectrometric imaging with enhanced positional resolution.

### Advantage of the Invention

According to another exemplary aspect of the invention, a matrix solution is provided, which is difficult to cause a plugging of a small size nozzle when the solution is discharged through the small size nozzle, while maintaining a sufficiently higher concentration of sinapinic acid frequently employed as a matrix in a sinapinic acid solution.

### BRIEF DESCRIPTION OF THE DRAWINGS

FIG. 1 is a graph, helpful in describing an advantageous effect of the present exemplary embodiment;

FIG. 2 is a graph, helpful in describing an advantageous effect of the present exemplary embodiment; and

FIG. 3 is a graph, showing results of the present example.

### BEST MODE FOR CARRYING OUT THE INVENTION

A matrix solution according to the present exemplary embodiment is a matrix solution to be discharged through a small size nozzle. The solution contains a liquid mixture consisting of acetonitrile and water as main solvent component, and a volumetric ratio of acetonitrile and water is within a range of from acetonitrile 6.5: water 3.5 to acetonitrile 8: water 2, and sinapinic acid is contained as the matrix.

The principal solvent component of the matrix solution according to the present exemplary embodiment is a liquid mixture of acetonitrile and water at a volumetric ratio of acetonitrile and water of within a range of from acetonitrile 6.5: water 3.5 to acetonitrile 8: water 2. The principal solvent component may be a constituent constituting equal to or higher than 90% of the solvent by volumetric ratio, and may more preferably be a constituent constituting equal to or higher than 97%.

The principal solvent component of the matrix solution according to the present exemplary embodiment may more desirably be a liquid mixture of acetonitrile and water at a volumetric ratio within a range of from acetonitrile 7: water 3 to acetonitrile 8: water 2.

The concentration of sinapinic acid is influenced by the sensibility of the mass spectrometry, and is not particularly limited provided that the concentration allows achieving the object of mass spectrometry, and preferably within a range of from 4.48 mg/mL to 11.2 mg/mL.

The matrix solution according to the present exemplary embodiment may contain a solvent, which is capable of being mixed with acetonitrile and water. A polar solvent such as alcohol and the like, for example, may be employed for such solvent.

An acid may be added to the matrix solution according to the present exemplary embodiment. Trifluoro acetic acid (TFA), for example, may be employed for such acid. While the concentration of acid is not particularly limited, the concentration may be, for example, within a range of from 0.1% to 1%, when TFA is employed.

The matrix solution according to the present exemplary embodiment is discharged through a small size nozzle. While the type of the small size nozzle is not particularly limited, typical example thereof may include an ink-jet, a dispenser, a spray, and an electrospray.

The matrix solution according to the present exemplary embodiment can be successfully discharged even if the solution is discharged through the small size nozzle having an internal diameter of the aperture of equal to or smaller than 0.18 mm. While the lower limit of the internal diameter of the aperture of the nozzle may not particularly limited provided that the aperture diameter is suitable for adding the matrix solution, the aperture diameter may be equal to or larger than 0.10 mm in view of a practicality, a commercial-availability and a cost.

While the temperature in the discharging process is not particularly limited provided that the temperature is suitable for the operation of the small size nozzle, it is preferable for discharging at a temperature within a range of from 10 to 30 degrees C., and it is more preferable for discharging at a temperature within a range of from 20 to 25 degrees C.

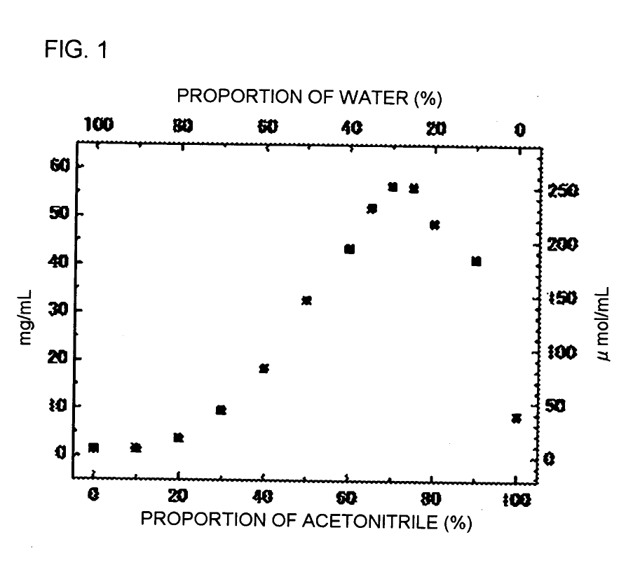

Advantageous effects obtainable by employing the configuration of the present exemplary embodiment will be described as follows. FIG. 1 is a graph which measured saturated concentration of sinapinic acid for solvent with room temperature for solvents of different mixing ratios of acetonitrile and water. Lower abscissa axis represents concentrations of acetonitrile, and upper abscissa axis represents concentrations of water, both in volumetric percent (%). Left ordinate axis represents concentrations of sinapinic acid in a unit of mg/mL, and right ordinate axis represents the converted molar concentrations equivalent to the concentration represented by the right ordinate axis in a unit of pmol/mL.

As shown in FIG. 1, the saturated concentration of sinapinic acid considerably varies with the mixing ratio of acetonitrile and water. The saturated concentration is maximized in the case of the volumetric ratio of acetonitrile 7 and water 3 with a maximum value of 56.4 mg/mL. The saturated concentrations in the cases of the volumetric ratio ranging from acetonitrile 6.5 and water 3.5 to acetonitrile 8 and water 2 are about 50 mg /mL or higher, which is equivalent to five folds of the saturated concentration of 10 mg/mL in the ordinary conditions. Therefore, sinapinic acid is difficult to precipitate by employing such condition of the volumetric ratio of acetonitrile and water when a solution of sinapinic acid is prepared at a concentration of 10 mg/mL, which is one-fifth of the saturated concentration, thereby preventing the needle from being plugged.

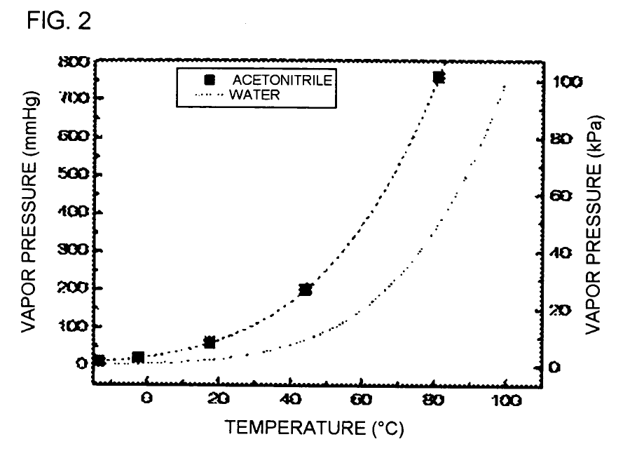

FIG. 2 is a graph, showing a temperature-vapor pressure curve of acetonitrile and water. In this graph, abscissa axis represents temperature (degree C.), and left ordinate axis represents vapor pressure in unit of mmHg and right ordinate axis represents the converted vapor pressure in unit of kPa.

As can be seen from the graph, acetonitrile exhibits a vapor pressure around 20 degrees C., which is about four folds of that of water. Therefore, the solvent evaporates in the gas-liquid interface at the tip of the small size nozzle according to such rate. The volumetric ratio of the principal solvent of the matrix solution according to the present exemplary embodiment is closer to the above-described ratio of the vapor pressure. Therefore, it may be understood that such volumetric ratio presents a stable condition, which is not much changed by the drying.

By summing up the conditions as described above, it is found that a matrix solution containing sinapinic acid dissolved as a matrix in a solvent prepared by mixing acetonitrile and water at a ratio within a range of from acetonitrile 7: water 3 to acetonitrile 8: water 2 is employed, so that the sinapinic acid solution of a concentration equivalent to the concentration employed in the conventional technology can be more stably dropped without plugging the small size nozzle.

In addition, the concentration of sinapinic acid in the solution may be equal to or higher than 4.48 mg/mL, namely equal to or higher than 20 pmol/μL for achieving stably measurements, which corresponds to two folds of the sample amount for the lower limit in the sensibility for the commercially available mass spectrometer of 10 fmol. On the other hand, the concentration may be equal to or lower than 11.2 mg/mL, which is not higher than fivefold of the saturated concentration. This allows adding sinapinic acid at a sufficient concentration for specimens serving as an object for the mass spectrometry and thereby preventing the needle from being plugged.

### Examples

### <Preparation of Matrix Solution>

Solvents for containing sinapinic acid dissolved therein were prepared by selecting the volumetric ratios of acetonitrile and water as acetonitrile/water=3:7, 6:4, 6.5:3.5, 7:3, 8:2, and 8.5:1.5. TFA of 0.1 to 1% is mixed into each of the solvents, and sinapinic acid is dissolved at a concentration of 10 mg/mL.

### <Experiments for Discharging Ability>

A metal needle for dispenser of 28G (internal diameter: 0.18 mm, external diameter: 0.36 mm) was employed to drop the respective matrix solutions prepared according to the above-described procedures at a room temperature to determine the discharging ability. In addition, matrix solutions were also dropped again after a certain time was past to determine the re-discharging ability for the matrix solutions.

### <Experiments for Solubility of Protein>

A protein of apomyoglobin was added to above-described prepared matrix solutions at a concentration of one-thousandth of the concentration of sinapinic acid in molar ratio to determine the solubility of apomyoglobin.

### <Experiments on Uniformity of Signal Detection Intensity>

A metal needle for dispenser of 32G (internal diameter: 0.10 mm, external diameter: 0.23 mm) was employed to respectively drop the above-described prepared matrix solutions containing apomyoglobin at a room temperature along a flow path of a chip substrate having a straight flow path.

The substrate was then transferred to the mass spectrometer for measuring the signal detection intensity of apomyoglobin to evaluate the uniformity of the signal detection intensity.

### <Results>

The results are shown in Table 1. The mark ◯ represents goodness, the mark X represents failure, and the mark Δ represents the conditions mixed up with goodness (◯) and failure (×) resulting in unstable condition.

TABLE 1

| | | | | | | | |
|---|---|---|---|---|---|---|---|
| VOLUMETRIC RATIO | ACETONITRILE | 3 | 6 | 6.5 | 7 | 8 | 8.5 |
| OF PRINCIPAL | WATER | 7 | 4 | 3.5 | 3 | 2 | 1.5 |
| SOLVENT OF MATRIX | | | | | | | |
| SOLUTION | | | | | | | |

| | | | | | | |
|---|---|---|---|---|---|---|
| DISCHARGING ABILITY | X | Δ | ◯ | ◯ | ◯ | ◯ |
| RE-DISCHARGING ABILITY | X | Δ | Δ | ◯ | ◯ | Δ |
| SOLUBILITY FOR PROTEIN | ◯ | ◯ | ◯ | ◯ | ◯ | X |
| UNIFORMITY OF SIGNAL | X | X | ◯ | ◯ | ◯ | — |
| DETECTION INTENSITY | | | | | | |

When the matrix solution of the ratio of acetonitrile/water of 3:7 was dropped in the experiments for the discharging ability, new crystals precipitated within one minutes after the crystals disposed at the tip of the needle was removed, plugging the needle. Further, a phenomenon of the needle being plugged in the dropping process was also observed. On the other hand, for the solutions in other volumetric ratios, the dropping processes were successfully carried out without causing a plugging of the needle.

The experiments for the re-discharging ability were repeatedly conducted, and it was found that stable discharging was exhibited for the solutions of the volumetric ratios of acetonitrile and water of 7:3 and 8:2.

In the experiments for the solubility of protein, while apomyoglobin was not well dissolved in a solvent with the volumetric ratio of acetonitrile and water of 8.5:1.5 due to the excessively higher ratio of the organic solvent, apomyoglobin was be able to be well dissolved in the matrix solutions of other volumetric ratios.

In the experiments for the uniformity of the signal detection intensity, the condition of the volumetric ratio of acetonitrile and water of 6:4 provides clearly deteriorated uniformity, as compared with the other conditions of the volumetric ratios. Then, the experiment was continued by diluting the solution to have the concentration of sinapinic acid of about 8.5 mg/mL, and the result showed that improved uniformity for the signal detection intensity was obtained. Therefore, it was found that the crystals more easily precipitate in the needle in the condition of the volumetric ratio of acetonitrile and water of 6:4, as compared with the other conditions of the volumetric ratio, easily causing unstable discharging rate of the solution. On the contrary, it was also found that the drying rate was lower and the solution more easily flowed in the condition of the volumetric ratio of acetonitrile and water of 6:4 as compared with other conditions of the volumetric ratios and thus the solution of such volumetric ratio was difficult to be applied to the mass spectrometric imaging.

In the condition of the volumetric ratio of acetonitrile and water of 6:4, the saturated concentration of sinapinic acid is 43 mg/mL from the graph of FIG. 1. In this condition, the concentration of sinapinic acid may be selected to be 8.5 mg/mL to obtain enhanced signal strength as described above, such that it was thought that the concentration of sinapinic acid should be not higher than 1/5 of the saturated concentration. Thus, solutions of matrix of sinapinic acid were prepared with the solvent with volumetric ratio of acetonitrile and water of 7:3 and 8:2 while calculating the concentration of the matrix by mol concentration, and experiments for the apomyoglobin detection sensitivity with a mass spectrometer were conducted with the prepared solutions of the matrix, and it was found that clear signal could be detected when the concentration of sinapinic acid was not lower than 4.48 mg/mL (20 pmol/μL).

The constitution of the matrix solution was optimized by such experiments to obtain the results, in which variations in the signal detection intensity for apomyoglobin were equal to or less than ±30% in the case of conducting the dropping operations along a flow path in a chip for the conditions of the volumetric ratio of /acetonitrile and water of 7:3 and 8:2. It was also confirmed that the detection was conducted within the flow path in the chip without disturbing a pattern of protein isolated by isoelectric focusing.

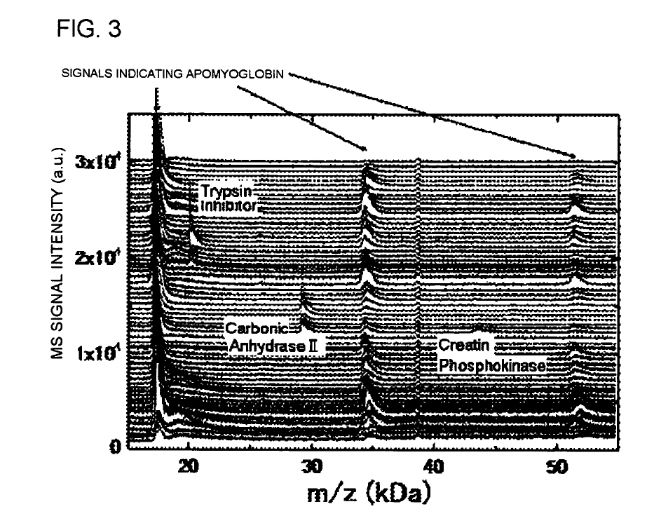

FIG. 3 is a graph, which includes mass spectrometry spectrums taken by every 0.5 mm in the flow path. Abscissa represents molecular weight, and ordinate represents signal strength. It is found that trypsin inhibitor, carbonic anhydrase II, and creatine phosphokinase, which are isolated in advance in the flow path in the chip by isoelectric focusing phoresis, were detected without particularly spreading through the inside of the flow path.

As described above, the matrix solution, which hardly causes a plugging of the small diameter nozzle during the dropping process through the nozzle while the concentration of the solution of sinapinic acid frequently employed as the matrix is maintained at the necessary and sufficient concentration, can be provided.

While the experimental results employing the dispenser is described in the present example, the configuration is not limited to the dispenser, and it is needless to point out that the configuration of the example may also be generally adopted to the technology for adding the matrix solution with the small diameter nozzle.

While the exemplary embodiments of the present invention have been described, it is intended to present these exemplary embodiments for the purpose of illustrations of the present invention only, and various modifications other than that described above are also available.

The present application claims priority based on Japanese patent application No. 2007-50,429 filed on 28 Feb. 2007, whole contents of which are hereby incorporated by reference.

1. A solution of a matrix for being discharged through a small size nozzle, containing a liquid mixture consisting of acetonitrile and water as main solvent component,
wherein a volumetric ratio of acetonitrile and water is within a range of from acetonitrile 6.5: water 3.5 to acetonitrile 8: water 2, and
wherein sinapinic acid is contained as the matrix.

2. The solution of the matrix as set forth in claim 1, wherein the volumetric ratio of acetonitrile and water is within a range of from acetonitrile 7: water 3 to acetonitrile 8: water 2.

3. The solution of the matrix as set forth in claim 1, wherein the concentration of sinapinic acid is within a range of from 4.48 mg/mL to 11.2 mg/mL.

4. The solution of the matrix as set forth in claim 1, wherein an inner diameter, of said small size nozzle is equal to or smaller than 0.18 mm.

5. The solution of the matrix as set forth in claim 1, wherein the solution is added to tissue slice, or a specimen separated by a chip or a gel.

6. A method for discharging a solution of a matrix through a small diameter nozzle, wherein the solution contains a liquid mixture consisting of acetonitrile and water as main solvent component,
wherein a volumetric ratio of acetonitrile and water is within a range of from acetonitrile 6.5: water 3.5 to acetonitrile 8: water 2, and
wherein sinapinic acid is contained as the matrix.

7. The method for discharging the solution of the matrix as set forth in claim 6, wherein the volumetric ratio of acetonitrile and water is within a range of from acetonitrile 7: water 3 to acetonitrile 8: water 2.

8. The method for discharging the solution of the matrix as set forth in claim 6, wherein the concentration of sinapinic acid is within a range of from 4.48 mg/mL to 11.2 mg/mL.

9. The method for discharging the solution of the matrix as set forth in claim 6, wherein an internal diameter of said small size nozzle is equal to or smaller than 0.18 mm.

10. The method for discharging the solution of the matrix as set forth in claim 6, wherein the solution is added to tissue slice, or a specimen separated by a chip or a gel.
